(19) United States
(12) Patent Application Publication
(10) Pub. No.: US 2013/0317040 A1
(43) Pub. Date: Nov. 28, 2013

(54) METHODS FOR TREATING SYSTEMIC LUPUS ERYTHEMATOSUS USING HIV PROTEASE INHIBITORS

(71) Applicants: Yousef Al-Abed, Dix Hills, NY (US); Betty A. Diamond, Bronx, NY (US)

(72) Inventors: Yousef Al-Abed, Dix Hills, NY (US); Betty A. Diamond, Bronx, NY (US)

(73) Assignee: The Feinstein Institute of Medical Research, Manhasset, NY (US)

(21) Appl. No.: 13/995,202

(22) PCT Filed: Dec. 21, 2011
(86) PCT No.: PCT/US11/66565

§ 371 Date: Aug. 13, 2013

Related U.S. Application Data
(60) Provisional application No. 61/426,221, filed on Dec. 22, 2010.

(51) Int. Cl. A61K 31/513; A61K 31/4418; A61K 31/34; A61K 31/427; A61K 31/496; A61K 31/472; A61K 31/4725

(52) U.S. Cl. CPC A61K 31/513; A61K 31/472; A61K 31/4418; A61K 31/4725; A61K 31/427; A61K 31/496; A61K 31/34
USPC 514/253.01; 514/307; 514/357; 514/274; 514/365; 514/470

(57) ABSTRACT

The present invention is directed to a method of treating systemic lupus erythematosus (SLE) in a subject comprising administering to the subject an amount of an HIV protease inhibitor effective to treat SLE. The present invention is also directed to a pharmaceutical product comprising an HIV protease inhibitor formulated in a pharmaceutically acceptable carrier, and a package insert providing instructions for the administration of the HIV protease inhibitor for the treatment of SLE. In addition, the present invention is directed to the use of an HIV protease inhibitor for the preparation of a medicament for the treatment of SLE.

### CROSS REFERENCE TO RELATED APPLICATIONS

The application claims the benefit of U.S. Provisional Application No. 61/426,221, filed Dec. 22, 2010, the content of which is hereby incorporated by reference it its entirety.

### FIELD OF THE INVENTION

The present invention relates to the treatment of systemic lupus erythematosus (SLE) using HIV protease inhibitors.

### BACKGROUND OF THE INVENTION

Systemic lupus erythematosus (SLE) is a chronic autoimmune disease affecting an estimated 5 million people worldwide, primarily young women. SLE is characterized by the presence of pathogenic autoantibodies, many of which are directed against nuclear antigens, in particular double stranded (ds) DNA. Clinical studies as well as animal models have shown that anti-dsDNA antibodies contribute to kidney disease and other complications of lupus. A subset of anti-dsDNA antibodies cross-reacts with the N methyl D aspartate receptor (NMDAR) on neurons and in the kidney. These autoantibodies are both neurotoxic and nephrotoxic and are present in approximately 30-40% of lupus patient sera and in cerebrospinal fluid (CSF) of patients with central nervous system (CNS) manifestations of SLE. The present invention addresses the need for small molecule compounds that can be used for the treatment of lupus.

### SUMMARY OF THE INVENTION

The present invention is directed to a method of treating systemic lupus erythematosus (SLE) in a subject comprising administering to the subject an amount of an HIV protease inhibitor effective to treat SLE.

The present invention is also directed to a pharmaceutical product comprising an HIV protease inhibitor formulated in a pharmaceutically acceptable carrier; and a package insert providing instructions for the administration of the HIV protease inhibitor for the treatment of systemic lupus erythematosus (SLE).

In addition, the present invention is directed to the use of an HIV protease inhibitor for the preparation of a medicament for the treatment of systemic lupus erythematosus (SLE). Additional objects of the invention will be apparent from the description which follows.

### DETAILED DESCRIPTION OF THE INVENTION

As discussed above, the present invention is directed to a method of treating systemic lupus erythematosus (SLE) in a subject comprising administering to the subject an amount of an HIV protease inhibitor effective to treat SLE.

As used herein, to treat SLE in a subject means to stabilize, reduce or eliminate a sign or symptom of SLE in the subject. SLE most often harms the heart, joints, skin, lungs, blood vessels, liver, kidneys, and nervous system. Subjects with SLE can experience fever, malaise, joint pains, myalgias, fatigue, and loss of cognitive abilities. SLE patients can suffer, for example, dermatological symptoms, such as the classic malar rash (or butterfly rash); hematological manifestations, such as anemia and iron deficiency and low platelet and white blood cell counts; inflammation of various parts of the heart, such as pericarditis, myocarditis, and endocarditis; lung and pleura inflammation; hematuria or proteinuria; and neuropsychiatric manifestation such as headache, cognitive dysfunction, mood disorder, cerebrovascular disease, seizures, polyneuropathy, anxiety disorder, and psychosis. The methods of treatment herein may effect prevention of an SLE flare, or reduce the extent or duration of an SLE flare, or reduce the frequency of SLE flares. In an embodiment, the SLE is treated by treating or reducing the extent of SLE-associated nephrotoxicity in the subject. In an embodiment, the SLE is treated by treating or reducing the extent of SLE-associated neurotoxicity in the subject. The methods disclosed herein may also be used to specifically effect treatment of a symptom of a renal lupus or of a symptom of a neuropsychiatric lupus.

As used herein, the HIV protease inhibitor can include but is not limited to commercially available HIV protease inhibitors such as nelfinavir, atazanavir, lopinavir, saquinavir, ritonavir, indinavir, darunavir, amprenavir, fosamprenavir and tipranavir, as well as derivatives and analogues of any of such compounds.

More specifically, the HIV protease used in the present invention can include atazanavir (Bristol Myers) or derivatives and analogues thereof such as the compounds described in U.S. Pat. No. 5,849,911, which is hereby incorporated by reference. Such compounds include but are not limited to 1-[4-(thiazol-5-yl)-phenyl]-4(S)-hydroxy-2-N—(N-methoxycartionyl-(L)-valyl)amino-5(S)—N—(N-methoxycarbonyl-p-tert-leucyl)amino-6-phenyl-2-azahexare; 1-[4-(thiazol-5-yl)-phenyl]-4(S)-hydroxy-2-N—(N-methoxycarbonyl-(L)-iso-leucyl)amino-5(S)—N—(N-methoxycarbonyl-(L)-tert-leucyl)amino-6-phenyl-2-azahexane; 1-[4-(thiazol-5-yl)-phenyl]-4(S)-hydroxy-2-N—(N-methoxycartonyl-(L)-S-methylcysteinyl)-amino-5(S)—N—(N-methoxycarbonyl-(L)-tert-leucyl)amino-6-phenyl-2-azahexane; 1-[4-(thiazol-5-yl)-phenyl]-4(S)-hydroxy-2-N—(N-ethoxycarboiyl-(L)-valyl)amino-5(S)—N—(N-methoxycarbonyl-(L)-tert-leucyl)amino-6-phenyl-2-azahexane; 1-[4-(thiazol-5-yl)-phenyl]-4(S)-hydroxy-2-N—(N-methoxycarbonyl-(L)-tert-leucyl)amino-5(S)—N—(N-methoxycarbonyl-(L)-valyl)amino-6-phenyl-2-azahexane; 1-[4-(thiazol-5-yl)-phenyl]-4(S)-hydroxy-2-N—(N-methoxycarbonyl-(L)-tert-leucyl)amino-5(S)—N—(N-methoxycarbonyl-(L)-iso-leucyl)amino-6-phenyl-2-azahexane; 1-[4-(thiazol-2-yl)-phenyl]-4(S)-hydroxy-5(S)-2,5-bis-N—(N-methoxycarbonyl-(L)-tert-leucyl)-amino-6-phenyl-2-azahexane; 1-[4-(thiazol-2-yl)-phenyl]-4(S)-hydroxy-2-N—(N-methoxycarbonyl-(L)-tert-leucyl)amino-5(S)—N—(N-methoxycarbonyl-β-valyl)amino-6-phenyl-2-azahexane; 1-[4-(thiazol-2-yl)-phenyl]-4(S)-hydroxy-2-N—(N-methoxycarbonyl-(L)-tert-leucyl)amino-5(S)—N—(N-methoxycarbonyl-(L)-iso-leucyl)amino-6-phenyl-2-azahexane; 1-[4-(pyridin-2-yl)-phenyl]-4(S)-hydroxy-5(S)-2,5-bis-[N—(N-methoxycarbonyl-(L)-valyl)amino]-6-phenyl-2-azahexane; 1-[4-(pyridin-2-yl)-phenyl]-4(S)-hydroxy-2-N—(N-methoxycarbonyl-(L)-valyl)amino-5(S)—N—(N-methoxycarbonyl-(L)-tert-leucyl)amino-6-phenyl-2-azahexane; 1-[4-(pyridin-2-yl)-phenyl]-4(S)-hydroxy-2-N—(N-methoxycarbonyl-p-tert-leucyl)amino-5(S)—N—(N-methoxycarbonyl-(L)-valyl)amino-6-phenyl-2-azahexane; 1-[4-(thiazol-5-yl)-phenyl]-4(S)-hydroxy-5(S)-2,5-bis-[N—(N-methoxycarbonyl-(L)-tert-leucyl)-amino]-6-phenyl-2-azahexane; 1-[4-(2-methyl-2H-tetrazol-5-yl)-phenyl]-4(S)-hydroxy-5(S)-2,5-bis-[N—(N-methoxycarbonyl-(L)-tert-leucyl)amino]-6-phenyl-2-azahexane; or 1-[4-(pyridin-2-yl)-phenyl]-4(S)-hydroxy-5(S)-2,5-bis-[N—(N-methoxycarbonyl-(L)-tert-leucyl)-amino]-6-phenyl-2-azahexane.

In another embodiment, the HIV protease can include lopinavir (Abbott) or derivatives and analogues thereof such as the compounds described in U.S. Pat. No. 5,914,332, which is hereby incorporated by reference. Such compounds include but are not limtied to (2S,3S,5S)-2-(2,6-dimethylphenoxyacetyl)amino-3-hydroxy-5-[2S-(1-tetrahydro-pyrimid-2-onyl)-3-methyl butanoyl]amino-1,6-diphenylhexane; (2S,3S,5S)-2-(2,6-Dimethylphenoxyacetyl)amino-3-hydroxy-5-(2S-(1-imidazolidin-2-onyl)-3,3-dimethyl butanoyl)amino-1,6-diphenylhexane; (2S,3S,5S)-2-(2,6-dimethylphenoxyacetyl)amino-3-hydroxy-5-(2S-(1-imidazolidin-2-thionyl)-3-methyl butanoyl)amino-1,6-diphenylhexane; (2S,3S,5S)-2-(2,4,6-trimethylphenoxyacetyl)amino-3-hydroxy-5-(2S-(1-imidazolidin-2-onyl-3-methylbutanoyl)amino-1,6-diphenylhexane; (2S,3S,5S)-2-(4-fluoro-2,6-dimethylphenoxy acetyl)amino-3-hydroxy-5-(2S-(1-imidazolidin-2-onyl)-3-methylbutanoyl)amino-1,6-diphenylhexane; (2S,3S,5S)-2-(trans-3-(2,6-dimethylphenyl)propenoyl)amino-3-hydroxy-5-(2S-1-tetrahydropyrimidin-2-onyl)-3-methyl-butanoyl)amino-1,6-diphenylhexane; (2S,3S,5S)-2-(3-(2,6-dimethylphenyl)propanoyl)amino-3-hydroxy-5-(2S-(1-tetrahydropyrimidin-2-onyl)-3-methyl-butanoyl)amino-1,6-diphenylhexane; or (2S,3S,5S)-2-(2,6-dimethylphenoxyacetyl)amino-3-hydroxy-5-(2S-(1-tetrahydro-pyrimid-2-onyl)-3-methylbutanoyl)amino-1-phenyl-6-methylheptane.

In another embodiment, the HIV protease can include saquinavir (Roche) or derivatives and analogues thereof such as the compounds described in U.S. Pat. No. 5,196,438, which is hereby incorporated by reference. Such compounds include but are not limtied to N-tert. butyl-decahydro-2-[2(R)-hydroxy-4-phenyl-3(S)-[[N-(2-quinolylcarbonyl)-L-asparaginyl]amino]butyl]-(4aS,8aS)-isoquinoline-3(S)-carboxamide; and N-tert.butyl-decahydro-2-[2(R)-hydroxy-4-phenyl-3(S)-[[N-benzyloxycarbonyl)-L-asparaginyl]amino]butyl]-(4aS,8aS)-isoquinoline-3(S)-carboxamide.

In yet another embodiment, the HIV protease can include ritonavir (Abbott) or derivatives and analogues thereof such as the compounds described in U.S. Pat. Nos. 5,541,206 and 5,648,497, which are hereby incorporated by reference. Such compounds include but are not limited to (2S,3S,5S)-5-(N—(N—((N-Methyl-N-((2-isopropyl-4-thiazolyl) methyl)amino)carbonyl)valinyl)amino)-2-(N-((5-thiazolyl)methoxycarbonyl)amino)-1,6-diphenyl-3-hydroxyhexane; (2S,3S,5S)-5-(N—(N—((N-Methyl-N-((2-isopropyl-4-thiazolyl) methyl)amino)carbonyl)alaninyl)amino)-2-(N-((5-thiazolyl) methoxycarbonyl)amino)-1,6-diphenyl-3-hydroxyhexane; (2S,3S,5S)-5-(N—(N-((2-isopropyl-4-thiazolyl)methoxy carbonyl) valinyl)amino)-2-(N-((5-thiazolyl)methoxy carbonyl)amino)-1,6-diphenyl-3-hydroxyhexane; (2S,3S,5S)-2-(N—(N-((2-Isopropyl-4-thiazolyl)methoxycarbonyl)valinyl)amino)-5-(N-((5-thiazolyl)methoxycarbonyl)amino)-1,6-diphenyl-3-hydroxyhexane; (2S,3S,5S)-5-(N—(N-((2-Isopropyl-4thiazolyl)methoxy carbonyl)alaninyl)amino)-2-(N-((5-thiazolyl)methoxy carbonyl)amino)-1,6-diphenyl-3-hydroxyhexane; (2S,3S,5S)-5-(N—(N-((2-(N,N-Dimethylamino)-4-thiazolyl)methoxycarbonyl)-valinyl)amino)-2-(N-((5-thiazolyl) methoxycarbonyl)amino)-1,6-diphenyl-3-hydroxyhexane; (2S,3S,5S)-2-(N—(N-((2-(N,N-Dimethylamino)-4-thiazolyl) methoxycarbonyl)-valinyl)amino)-5-(N-((5-thiazolyl)methoxycarbonyl)amino)-1,6-diphenyl-3-hydroxyhexane; (2S,3S,5S)-5-(N—(N-((2-(4-morpholinyl)-4-thiazolyl)methoxycarbonyl) valinyl)amino)-2-(N-((5-thiazolyl)methoxycarbonyl)amino)-1,6-diphenyl-3-hydroxyhexane; (2S,3S,5S)-2-(N—(N-((2-(4-Morpholinyl)-4-thiazolyl)-methoxycarbonyl)valinyl)-amino)-5-(N-((5-thiazolyl)-methoxy carbonyl)amino)-1,6-diphenyl-3-hydroxyhexane; (2S,3S,5S)-5-(N—(N-((2-(1-Pyrrolidinyl)-4-thiazolyl)methoxycarbonyl)valinyl)amino)-2-(N-((5-methoxycarbonyl)amino-1,6-diphenyl-3-hydroxyhexane; (2S,3S,5S)-5-(N—(N—((N-Methyl-N-((2-isopropyl-4-oxazolyl)methyl)amino)carbonyl)valinyl)amino)-2-(N-((5-thiazolyl)methoxycarbonyl)amino-1,6-diphenyl-3-hydroxyhexane; (2S,3S,5S)-5-(N—(N—((N-Methyl-N-((2-isopropyl-4-oxazolyl) methyl)amino)carbonyl)valinyl)amino)-2-(N-((5-oxazolyl)methoxycarbonyl)amino)-1,6-diphenyl-3-hydroxyhexane; (2S,3S,5S)-5-(N—(N—((N-Methyl-N-((2-isopropyl-4-thiazolyl)methyl)amino)-carbonyl)valinyl)amino)-2-(N-((5-oxazolyl)methoxycarbonyl)amino)-1,6-diphenyl-3-hydroxyhexane; (2S,3S,5S)-5-(N—(N—((N-Methyl-N-((2-isopropyl-4-thiazolyl)methyl)amino)carbonyl)valinyl)amino)-2-(N-((5-isoxazolyl)methoxycarbonyl)amino)-1,6-diphenyl-3-hydroxyhexane; or (2S,3S,5S)-5-(N—(N4N-Methyl-N-((2-isopropyl-4-oxazolyl)methyl)amino)carbonyl)valinyl)amino)-2-(N-((5-isoxazolyl)methoxycarbonyl)amino)-1,6-diphenyl-3-hydroxyhexane.

In yet another embodiment, the HIV protease can include indinavir (Merck) or derivatives and analogues thereof such as the compounds described in U.S. Pat. No. 5,413,999, which is hereby incorporated by reference. Such compounds include but are not limited to N-(2(R)-hydroxy-1(S)-indanyl)-2(R)-phenylmethyl-4(S)-hydroxy-5-(2-(3-(S)—N′-(t-butylcarboxamido)-(4aS,8aS)-decahydroisoquinoline)yl)-pentaneamide; N-(2(R)-hydroxy-1(S)-indanyl)-2(R)-phenylmethyl(4(S)-hydroxy-5-(1-(4-carbobenzyloxy-2(S)—N′-(t-butylcarboxamido)-piperazinyl))-pentaneamide; N-(2(R)-hydroxy-1(S)-indanyl)-2(R)-((4-(2-(4-morpholinyl)ethoxy)phenyl)methyl)-4(S)-hydroxy-5-(2-(3(S)—N′-(t-butylcarboxamido)-(4aS,8aS)-decahydroisoquinoline)yl)-pentaneamide; N-(2(R)-hydroxy-1(S)-indanyl)-2(R)-((4-(2-(4-morpholinyl)ethoxy)phenyl)methyl-4(S)-hydroxy-5-(1-(4-carbobenzyloxy-2(S)—N′-(t-butylcarboxamido)-piperazinyl))-pentaneamide; N-(2(R)-hydroxy-1(S)-indanyl)-2(R)-((4-((2-hydroxy)-ethoxy)phenyl)methyl)-4(S)-hydroxy-5-(2-(3(S)—N′-(t-butylcarboxamido)-(4aS,8aS)-decahydroisoquinoline)-yl)-pentaneamide; N-(2(R)-hydroxy-1(S)-indanyl)-2(R)-((4-((2-hydroxy)-ethoxy)phenyl)methyl)-4(S)-hydroxy-5-(1-(4-carbobenzyloxy-2(S)—N′-(t-butylcarboxamido)-piperazinyl))-pentaneamide; N-(4(S)-3,4-dihydro-1H-2,2-dioxobenzothiopyranyl)-2(R)-phenylmethyl-4(S)-hydroxy-5-(2-(3(S)—N′-(t-butyl carboxamido)-(4aS,8aS)-decahydroisoquinoline)yl)-pentaneamide; N-(4(S)-3,4-dihydro-1H-2,2-dioxobenzothio pyranyl)-(2(R)-phenylmethyl-4(S)-hydroxy-5-(1-(4-carbobenzyloxy-2(S)—N′-(t-butyl carboxamido)-piperazinyl))-pentaneamide; N-(2(R)-hydroxy-1(S)-indanyl)-2(R)-phenylmethyl-4(S)-hydroxy-5-(1-(4-(3-pyridylmethyl)-2(S)—N(t-butylcarboxamido)-piperazinyl))-pentaneamide; N-(2(R)-hydroxy-1(S)-indanyl)-2(R)-phenylmethyl-4-(S)-hydroxy-5-(1-(N-(t-butyl)-4(S)-phenoxyprolineamid)yl)-pentaneamide; N-(2(R)-hydroxy-1(S)-indanyl)-2(R)-phenylmethyl-4-(S)-hydroxy-5-(1-(N′-t-butyl-4(S)-2-naphthyloxy-prolineamid)yl)-pentaneamide; N-(2(R)-hydroxy-1(S)— indanyl)-2(R)-phenylmethyl-4-(S)-hydroxy-5-(1-(N′-t-butyl-4(S)-1-naphthyloxy-prolineamid)yl)-pentaneamide; N-(2(R)-hydroxy-1(S)-indanyl)-2(R)-phenylmethyl-4-(S)-amino-5-(2-(3(S)—N-(t-butylcarboxamido)-(4aS, 8a S)-decahydroisoquinoline)yl)-pentaneamide; N-(2(R)-hydroxy-1(S)-indanyl)-2(R)-phenylmethyl-4-(S)-hydroxy-5-(1-(4-(3-phenylpropionyl)-2(S)—N′-(t-butylcarboxamido)-piperazinyl))-pentaneamide; N-(2(R)-hydroxy-1(S)-indanyl)-2(R)-phenylmethyl-4-(S)-hydroxy-5-(1-(4-benzoyl-2(S)—N′-(t-butylcarboxamido)-piperazinyl))-pentaneamide; N-(2(R)-hydroxy-1(S)-indanyl)-2(R)-phenylmethyl-4-(S)-hydroxy-5-(1-(4-(3-phenylpropyl)-2(S)—N′-(t-butylcarboxamido)-piperazinyl))-pentaneamide; N-(2(R)-hydroxy-1(S)-indanyl)-2(R)-phenylmethyl-4-(S)-amino-5-(1-(4-carbobenzyloxy-2(S)—N-(t-butylcarboxamido)-piperazinyl))-pentaneamide; N-(2(R)-hydroxy-1(S)-indanyl)-2(R)-((4-(2-(4-morpholinyl) ethoxy)phenyl)methyl)-4(S)-hydroxy-5-(1-(N-(t-butyl)-4(S)-phenoxyprolineamid)yl)-pentaneamide; N-(2(R)-hydroxy-1(S)-indanyl)-2(R)-((4-(2-(4-morpholinyl)ethoxy)phenyl)methyl)-4(S)-hydroxy-5-(1-(N′-t-butyl-4(S)-2-naphthyloxy-prolineamid)yl)-pentaneamide; N-(2(R)-hydroxy-1(S)-indanyl)-2(R)-((4-(2-(4-morpholinyl)ethoxy)phenyl)methyl)-4(S)-hydroxy-5-(1-(N′-t-butyl-4(S)-1-naphthyloxy-prolineamid)yl)-pentaneamide; N-(2(R)-hydroxy-1(S)-indanyl)-2(R)-((4-(2-(4-morpholinyl)ethoxy)phenyl)methyl)-4(S)-amino-5-(2-(3(S)—N-(t-butylcarboxamido)-(4aS,8aS)-decahydroisoquinoline)yl)-pentaneamide; N-(2(R)-hydroxy-1(S)-indanyl)-2(R)-((4-(2-(4-morpholinyl)ethoxy)phenyl)methyl)-4(S)-hydroxy-5-(1-(4-(3-phenylpropionyl)-2 (S)—N′-(t-butylcarboxamido)-piperazinyl))-pentaneamide; N-(2(R)-hydroxy-1(S)-indanyl)-2(R)-((4-(2-(4-morpholinyl)ethoxy)phenyl)methyl)-4(S)-hydroxy-5-(1-(4-benzoyl-2(S)—N′-(t-butylcarboxamido)-piperazinyl))-pentaneamide; N-(2(R)-hydroxy-1(S)-indanyl)-2(R)-((4-(2-(4-morpholinyl)ethoxy)phenyl)methyl)-4(S)-hydroxy-5-(1-(4-(3-phenylpropyl)-2(S)—N′-(t-burylcarboxamido))-piperazinyl)-pentaneamide; N-(2(R)-hydroxy-1(S)-indanyl)-2(R)-((4-(2-(4-morpholinyl)ethoxy)phenyl)methyl)-4(S)-amino-5-(1-(4-carbobenzyloxy-2(S)—N-(t-butylcarboxamido)piperazinyl)pentaneamide; N-(2(R)-hydroxy-1(S)-indanyl)-2(R)-((4-((2-hydroxy)-ethoxy)phenyl)methyl)-4(S)-hydroxy-5-(1-(N′-(t-butyl)-4(S)-phenoxyprolineamid)yl)-pentaneamide; N-(2(R)-hydroxy-1(S)-indanyl)-2(R)-((4-((2-hydroxy)ethoxy)phenyl)methyl)-4(S)-hydroxy-5-(1-(N′-t-butyl-4(S)-2-naphthyloxy-prolineamid)yl)-pentaneamide; N-(2(R)-hydroxy-1(S)-indanyl)-2(R)-((4-((2-hydroxy)-ethoxy)phenyl)methyl)-4(S)-hydroxy-5-(1-(N′-t-butyl-4(S)-1-naphthyloxy-prolineamid)yl)-pentaneamide; N-(2(R)-hydroxy-1(S)-indanyl)-2(R)-((4-((2-hydroxy)ethoxy)phenyl)methyl)-4(S)-amino-5-(2-(3(S)—N′-(t-butylcarboxamido)-(4aS,8aS)-decahydroisoquinoline)-yl)pentaneamide; N-(2(R)-hydroxy-1(S)-indanyl)-2(R)-((4-((2-hydroxy)-ethoxy)phenyl)methyl)-4(S)-hydroxy-5-(1-(4-(3-phenylpropionyl)-2(S)—N′-(t-butylcarboxamido)-piperazinyl))pentaneamide; N-(2(R)-hydroxy-1(S)-indanyl)-2(R)-((4-((2-hydroxy)-ethoxy)phenyl)methyl)-4(S)-hydroxy-5-(1-(4-benzoyl2(S)—N′-(t-butylcarboxamido)-piperazinyl))-pentaneamide; N-(2(R)-hydroxy-1(S)-indanyl)-2(R)-((4-((2-hydroxy)-ethoxy)phenyl)methyl)-4(S)-hydroxy-5-(1-(4-(3-phenylpropyl)-2 (S)—N′-(t-butylcarboxamido))-piperazinyl)-pentaneamide; N-(2(R)-hydroxy-1(S)-indanyl)-2(R)-((4-((2-hydroxy)-ethoxy)phenyl)methyl)-4(S)-amino-5-(1-(4-carbobenzyloxy-2(S)—N-(t-butylcarboxamido)-piperazinyl))-pentaneamide; N-(4(S)-3,4-dihydro-1H-2,2-dioxobenzothiopyranyl)-2(R)-phenylmethyl-4(S)-hydroxy-5-(1-(N′-(t-butyl)-4(S)-phenoxyprolineamid)yl)-pentaneamide; N-(4(S)-3,4-dihydro-1H-2,2-dioxobenzothiopyranyl)-2-(R)-phenylmethyl-4(S)-hydroxy-5-(1-(N′-t-butyl-4(S)-2-naphthyloxy-prolineamid)yl)-pentaneamide; N-(4(S)-3,4-dihydro-1H-2,2-dioxobenzothiopyranyl)-2-(R)-phenylmethyl-4(S)-hydroxy-5-(1-(N′-t-butyl-4(S)-1-naphthyloxy-prolineamid)yl)-pentaneamide; N-(4(S)-3,4-dihydro-1H-2,2-dioxobenzothiopyranyl)-2-(R)-phenylmethyl-4(S)-amino-5-(2-(3(S)—N-(t-butylcarboxamido)-(4aS,8aS)-decahydroisoquinoline)yl)pentaneamide; N-(4(S)-3,4-dihydro-1H-2,2-dioxobenzothiopyranyl)-2-(R)-phenylmethyl-4(S)-hydroxy-5-(1-(4-(3-phenylpropionyl)-2(S)—N′-(t-butylcarboxamido)-piperazinyl))pentaneamide; N-(4(S)-3,4-dihydro-1H-2,2-dioxobenzothiopyranyl)-2-(R)-phenylmethyl-4(S)-hydroxy-5-(1-(4-benzoyl-2(S)—N′-(t-butylcarboxamido)-piperazinyl))-pentaneamide; N-(4(S)-3,4-dihydro-1H-2,2-dioxobenzothiopyranyl)-2-(R)-phenylmethyl-4(S)-hydroxy-5-(1-(4-(3-phenylpropyl)-2 (S)—N-(t-butylcarboxamido))-piperazinyl)pentaneamide, or (4(S)-3,4-dihydro-1H-2,2-dioxobenzothiopyranyl)-2-(R)-phenylmethyl-4(S)-amino-5-(1-(4-carbobenzyloxy-2-(S)N′-(t-butylcarboxamido)-piperazinyl))-pentaneamide.

In yet another embodiment, the HIV protease can include darunavir (Tbotec) or derivatives and analogues thereof such as the compounds described in U.S. Pat. No. 5,843,946, which is hereby incorporated by reference. Such compounds include but are not limited to phenylmethyl[2R-hydroxy-3-[(2-methylpropyl)(phenylsulfonyl)amino]-1S-(phenylmethyl)propyl]carbamate; phenylmethyl[2R-hydroxy-3-[(2-methylpropyl)(4-methoxyphenylsulfonyl)amino]-1S-(phenylmethyl)propyl]carbamate; phenylmethyl[2R-hydroxy-3-[(2-methylpropyl)(4-fluorophenylsulfonyl)amino]-1S-(phenylmethyl)propyl]carbamate; phenylmethyl[2R-hydroxy-3-[(2-methylpropyl)(4-nitrophenylsulfonyl)amino]-1S-(phenylmethyl)propyl]carbamate; phenylmethyl[2R-hydroxy-3-[(2-methylpropyl)(4-chlorophenylsulfonyl)amino]-1S-(phenylmethyl)propyl]carbamate; phenylmethyl[2R-hydroxy-3-[(2-methylpropyl)(4-acetamidophenylsulfonyl)amino]-1S-(phenylmethyl)propyl]carbamate; phenylmethyl[2R-hydroxy-3-[(2-methylpropyl)(4-aminophenylsulfonyl)amino]-1S-(phenylmethyl)propyl]carbamate; phenylmethyl[2R-hydroxy-3-[(3-methylbutyl)(4-methoxyphenyl sulfonyl)amino]-1S-(phenylmethyl)propyl]carbamate; phenylmethyl[2R-hydroxy-3-[(3-methylbutyl)(4-fluorophenylsulfonyl)amino]-1S-(phenylmethyl)propyl]carbamate; phenylmethyl[2R-hydroxy-3-[(3-methylbutyl)(4-nitrophenylsulfonyl)amino]-1S-(phenylmethyl)propyl]carbamate; phenylmethyl[2R-hydroxy-3-[(3-methylbutyl)(4-chlorophenylsulfonyl)amino]-1S-(phenylmethyl)propyl]carbamate; phenylmethyl[2R-hydroxy-3-[(2-methylpropyl)(4-methoxyphenylsulfonyl)amino]-1S-(4-fluorophenylmethyl) propyl]carbamate; phenylmethyl[2R-hydroxy-3-[(2-methylpropyl)(4-fluorophenylsulfonyl)amino]-1S-(4-fluoro phenylmethyl)propyl]carbamate; phenylmethyl[2R-hydroxy-3-[(butyl)(phenylsulfonyl)amino]-1S-(phenylmethyl)propyl]carbamate; phenylmethyl[2R-hydroxy-3-[(cyclohexylmethyl) (phenylsulfonyl)amino]-1S-(phenylmethyl)propyl]carbamate; phenylmethyl[2R-hydroxy-3-[(cyclohexyl)(phenylsulfonyl)amino]-1S-(phenylmethyl)propyl]carbamate; phenylmethyl[2R-hydroxy-3-[(propyl)(phenylsulfonyl)amino]-1S-(phenylmethyl) propyl]carbamate; 2S-[[(dimethylamino)acetyl]amino]-N-2R-hydroxy-3-[(3-methylpropyl)(4-methoxyphenylsulfonyl)amino]-1S-(phenylmethyl)propyl]-3S-methylpentanamide; 2S-[[(methylamino)acetyl]amino]-N-2R-hydroxy-3-[(4-methylbutyl)(phenylsulfonyl)amino]-1S-(phenylmethyl)propyl]-3S-methylpentanamide; 2S-[[(dimethylamino)acetyl]amino]-N-2R-hydroxy-3-[(4-methylbutyl)(phenylsulfonyl)amino]-1S-(phenylmethyl)propyl]-3S-methyl pentanamide; N-[2R-hydroxy-3-[[(4-methoxyphenyl)sulfonyl](2-methylpropyl)amino]-1S-(phenylmethyl)propyl]-4-pyridinecarboxamide; N-[2R-hydroxy-3-[[(4-methoxyphenyl) sulfonyl](2-methylpropyl)amino]-1S-(phenylmethyl)propyl]-2,6-dimethylbenzamide; N-[2R-hydroxy-3-[[(4-methoxyphenyl)sulfonyl](2-methylpropyl)amino]-1S-(phenylmethyl)propyl]-2-methylbenzamide; N-[2R-hydroxy-3-[[(4-methoxyphenyl)sulfonyl](2-methylpropyl)amino]-1S-(phenylmethyl)propyl]-2-ethylbenzamide; N-[2R-hydroxy-3-[[(4-methoxyphenyl)sulfonyl](2-methylpropyl)amino]-1S-(phenylmethyl)propyl]-2-chlorobenzamide; [2R-hydroxy-3-[[(4-methoxyphenyl)sulfonyl](2-methylpropyl)amino]-1S-(phenylmethyl)propyl]carbamic acid, 3-pyridylmethyl ester; [2R-hydroxy-3-[[(4-methoxy phenyl)sulfonyl](2-methylpropyl)amino]-1S-(phenylmethyl)propyl]carbamic acid, 3-pyridylmethyl ester, N-oxide; [2R-hydroxy-3-[[phenylsulfonyl](2-methylpropyl)amino]-1S-(phenylmethyl)propyl]carbamic acid, 3-pyridyl methyl ester; [2R-hydroxy-3-[[(4-methoxyphenyl)sulfonyl](2-methylpropyl)amino]-1S-(phenylmethyl)propyl]carbamic acid, 4-pyridylmethyl ester; [2R-hydroxy-3-[[(4-methoxy phenyl)sulfonyl](2-methylpropyl)amino]-1S-(phenylmethyl)propyl]carbamic acid, 4-pyridylmethyl ester, N-oxide; [2R-hydroxy-3-[[(4-chlorophenyl)sulfonyl](2-methylpropyl)amino]-1S-(phenylmethyl)propyl]carbamic acid, 3-pyridylmethyl ester; [2R-hydroxy-3-[[(4-nitrophenyl)sulfonyl](2-methylpropyl)amino]-1S-(phenylmethyl)propyl]carbamic acid, 3-pyridylmethyl ester; [2R-hydroxy-3-[[(4-fluorophenyl)sulfonyl](2-methylpropyl)amino]-1-S-(phenylmethyl)propyl]carbamic acid, 3-pyridylmethyl ester; [2R-hydroxy-3-[[(4-hydroxy phenyl)sulfonyl](2-methylpropyl)amino]-1S-(phenylmethyl)propyl]carbamic acid, 3-pyridylmethyl ester; or [2R-hydroxy-3-[[(4-methoxyphenyl)sulfonyl](2-methylpropyl)amino]-1S-(phenylmethyl)propyl]carbamic acid, 5-pyrimidylmethyl ester.

In yet another embodiment, the HIV protease can include nelfinavir (Agouron) or derivatives and analogues thereof such as the compounds described in U.S. Pat. No. 5,484,926, which is hereby incorporated by reference. Such compounds include but are not limited to 2-[2′-hydroxy-3′-phenylthiomethyl-4′-aza-5′-oxo-5′-(2″-methyl-3″-hydroxyphenyl) pentyl]decahydro isoquinoline-3-N-t-butylcarboxamide; 2-[2′-hydroxy-3′-phenylthiomethyl-4′-aza-5′-oxo-5′-(2″-methyl-3″-hydroxyphenyl)pentyl]decahydroisoquinoline-3-N-t-butyl carboxamide methanesulfonic acid salt; 2-[2′-hydroxy-3′-phenylthiomethyl-4′-aza-5′-oxo-5′-(2″-methyl-3″-hydroxyphenyl)pentyl]decahydroisoquinoline-3-N-t-butylcarboxamide 3″-dihydrogen phosphate hydrochloride salt; 2-[2′-hydroxy-3′-phenylthiomethyl-4′aza-5′-oxo-5′-(2″-methyl-3″-hydroxyphenyl)pentyl]-octahydro-thieno[3,2-c]pyridine-6-N-t-butyl carboxamide; and 2-[2′-hydroxy-3′-phenylthiomethyl-4′aza-5′-oxo-5′-(2″-methyl-3″-hydroxyphenyl)pentyl]-octahydro-thieno[3,2-c]pyridine-6-N-t-butylcarboxamide methane sulfonic acid salt. In an embodiment, the nelfinavir has the structure:

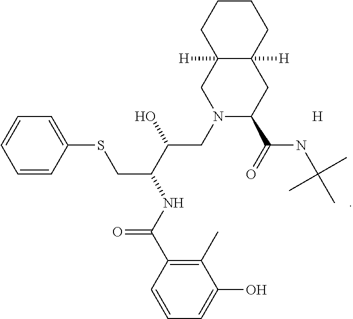

In an embodiment, nelfinavir is the mesylate salt and is [3S[2(2S*, 3S*), 3α,4β,8aβ]]-N-(1,1-dimethylethyl)decahydro-2-[2-hydroxy-3-[(3-hydroxy-2-methylbenzoyl)amino]-4-(phenylthio)butyl]-3-isoquinoline carboxamide mono-methanesulfonate.

In yet another embodiment, the HIV protease can include amprenavir (GSK) or derivatives and analogues thereof such as the compounds described in U.S. Pat. No. 5,585,397, which is hereby incorporated by reference. Such compounds include but are not limited to 4-Fluoro-N-((2 syn,3S)-2-hydroxy-4-phenyl-3-((S)-tetrahydrofuran-3-yloxy carbonylamino)-butyl)-N-isobutyl-benzenesulfonamide; 3,4-Dichloro-N-((2 syn,3S)-2-hydroxy-4-phenyl-3-((S)-tetrahydrofuran-3-yloxycarbonylamino)-butyl)-N-isobutyl-benzenesulfonamide; N-(4-(((2 syn,3S)-2-Hydroxy-4-phenyl-3-((S)-tetrahydrofuran-3-yloxycarbonylamino)-butyl)-isobutyl-sulfamoyl)-phenyl)-acetamide; 4-Fluoro-N-((2 syn,3S)-2-hydroxy-4-phenyl-3-((R)-tetrahydrofuran-3-yloxycarbonylamino)-butyl)-N-isobutyl-benzenesulfonamide and 4-Fluoro-N-((2 syn,3S)-2-hydroxy-4-phenyl-3-((R)-tetrahydrofuran-3-yloxycarbonylamino)-butyl)-N-isobutyl-benzenesulfonamide; N-(4-(((2 syn,3S)-2-Hydroxy-4-phenyl-3-((R)-tetrahydrofuran-3-yloxycarbonylamino)-butyl)-isobutyl-sulfamoylphenyl)-acetamide and N-(4-(((2 syn,3S)-2-Hydroxy-4-phenyl-3-((S)-tetrahydrofuran-3-yloxy carbonylamino)-butyl)-isobutyl-sulfamoyl)-phenyl)-acetamide; N-(2-Fluoro-5-(((2 syn,3S)-2-hydroxy-4-phenyl-3-((S)-tetrahydrofuran-3-yloxycarbonylamino)-butyl)-isobutyl-sulfamoyl)-phenyl)-acetamide; N-(3-(((2 syn,3S)-2-Hydroxy-4-phenyl-3-((S)-tetrahydrofuran-3-yloxycarbonylamino)-butyl)-isobutyl-sulfamoyl)-phenyl)-acetamide; 4-Fluoro-N-((2 syn,3S)-2-hydroxy-4-phenyl-3-((R)-tetrahydrofuran-3-yloxycarbonylamino)-butyl)-N-isobutyl-benzenesulfonamide; N-(4-(((syn)-2-Hydroxy-(S)-4-phenyl-3-((tetrahydro-furan-(R)-3-yl)-oxycarbonylamino)-butyl)-isobutyl-sulfamoyl)-phenyl)-acetamide; 4-Chloro-N-((2 syn,3S)-2-hydroxy-4-phenyl-3-((S)-tetrahydrofuran-3-yloxycarbonylamino)-butyl)-isobutyl-benzene sulfonamide; N-((2 syn,3S)-2-Hydroxy-4-phenyl-3-((S)-tetrahydrofuran-3-yloxy carbonyl amino)-butyl)-N-isobutyl-4-methoxy-benzenesulfonamide; Benzene-1,3-disulfonic acid 1-amide 3-((2 syn,3 S)-2-hydroxy-4-phenyl-3-(3-(S)-tetrahydrofuran-3-yloxycarbonylamino)-butyl)-isobutyl-amide; 4-Chloro-N-cyclopentylmethyl-N-((2 syn,3S)-2-hydroxy-4-phenyl-3-((S)-tetrahydrofuran-3-yloxycarbonylamino)-butyl)-benzenesulfonamide; N-(4-(cyclopentylmethyl-((2 syn,3S)-2-hydroxy-4-phenyl-3-((S)-tetrahydrofuran-3-yloxycarbonylamino)-butyl)-sulfamoyl)-phenyl)-acetamide; 3-chloro-N-((2 syn,3S)-2-hydroxy-4-phenyl-3-((S)-tetrahydro furan-3-yloxycarbonylamino)-butyl)-N-isobutyl-benzenesulfonamide; N-cyclopentylmethyl-N-((2 syn,3S)-2-hydroxy-4-phenyl-3-((S)-tetrahydrofuran-3-yloxycarbonylamino)-butyl)-4-methoxy-benzenesulfonamide; N-cyclopentylmethyl-N-((2 syn,3S)-2-hydroxy-4-phenyl-3-((S)-tetrahydrofuran-3-yloxycarbonylamino)-butyl)-benzenesulfonamide; N-cyclohexyl methyl-N-((2 syn,3S)-2-hydroxy-4-phenyl-3-((S)-tetrahydrofuran-3-yloxycarbonylamino)-butyl)-4-methoxy-benzenesulfonamide; N-cyclohexylmethyl-4-fluoro-N-((2 syn,3S)-2-hydroxy-4-phenyl-3-((S)-tetrahydrofuran-3-yloxycarbonylamino)-butyl)-benzenesulfonamide; N-(4-(cyclohexylmethyl)-((2 syn,3S)-2-hydroxy-4-phenyl-3-((S)-tetrahydrofuran-3-yloxycarbonylamino)-butyl)-sulfamoylphenyl)-acetamide: N-((2 syn,3 S)-2-Hydroxy-4-phenyl-3-((syn)-tetrahydrofuran-3-yloxycarbonylamino)-butyl)-N-isobutyl-4-methyl-benzene sulfonamide; N-((2 syn,3S)-2-Hydroxy-4-phenyl-3-((S)-tetrahydrofuran-3-yloxycarbonyl amino)-butyl)-N-isobutyl-4-nitro-benzenesulfonamide; 4-amino-N-((2 syn,3S)-2-Hydroxy-4-phenyl-3-((S)-tetrahydrofuran-3-yloxycarbonylamino)-butyl)-N-isobutyl-benzenesulfonamide; N-Cyclopentylmethyl-4-hydroxy-N-((2 syn,3S)-2-hydroxy-4-phenyl-3-((S)-tetrahydrofuran-3-yloxycarbonylamino)-butyl)-benzenesulfonamide; N-cyclopentylmethyl-N-((2 syn,3S)-2-hydroxy-4-phenyl-3-((S)-tetrahydrofuran-3-yloxycarbonylamino)-butyl)-4-nitro-benzenesulfonamide; 4-Amino-N-cyclopentylmethyl-N-((2 syn,3S)-2-hydroxy-4-phenyl-3-((S)-tetrahydrofuran-3-yloxycarbonylamino)-butyl)-benzenesulfonamide; 2,4-Diamino-N-cyclopentylmethyl-N-((2 syn,3S)-2-hydroxy-4-phenyl-3-((S)-tetrahydrofuran-3-yloxycarbonyl amino)-butyl)-benzenesulfonamide; 4-Hydroxy-N-(2 syn,3S)-2-hydroxy-4-phenyl-3-((S)-tetrahydrofuran-3-yloxycarbonylamino)-butyl)-N-isobutyl-benzenesulfonamide; N-cyclopentylmethyl-4-fluoro-N-((2 syn,3S)-2-hydroxy-4-phenyl-3-((S)-tetrahydrofuran-3-yloxycarbonylamino)-butyl)-benzenesulfonamide; and 3,4-dichloro-N-cyclopentylmethyl-N-((2 syn,3S)-2-hydroxy-4-phenyl-3-((S)-tetrahydrofuran-3-yloxycarbonylamino)-butyl)-benzenesulfonamide.

In yet another embodiment, the HIV protease can include a prodrug of amprenavir such as fosamprenavir (GSK) or derivatives and analogues thereof such as the compounds described in U.S. Pat. No. 6,436,989, which is hereby incorporated by reference.

In yet another embodiment, the HIV protease can include tipranavir (Boehringer Ingelheim) or derivatives and analogues thereof such as the compounds described in U.S. Pat. No. 5,852,195, which is hereby incorporated by reference. Such compounds include but are not limited to 5-trifluoromethyl-N-[3-(R or S)-[1-[4-hydroxy-2-oxo-6,6-di-n-propyl-5,6-dihydro-2H-pyran-3-yl]-propyl]-phenyl]-2-pyridinesulfonamide; 5-trifluoromethyl-N-[3-(R)-[1-[4-hydroxy-2-oxo-6,6-di-n-propyl-5,6-dihydro-2H-pyran-3-yl]-propyl]-phenyl]-2-pyridine sulfonamide; (3R)—N-[3-[1-(5,6-Dihydro-4-hydroxy-2-oxo-6,6-dipropyl-2H-pyran-3-yl)propyl]phenyl]-5-(trifluoromethyl)-2-pyridinesulfonamide; 5-trifluoromethyl-N-[3-(S)-[1-[4-hydroxy-2-oxo-6,6-di-n-propyl-5,6-dihydro-2H-pyran-3-yl]-propyl]-phenyl]-2-pyridinesulfonamide; (3S)—N-[3-[1-(5,6-dihydro-4-hydroxy-2-oxo-6,6-dipropyl-2H-pyran-3-yl)propyl]phenyl]-5-(trifluoromethyl)-2-pyridinesulfonamide; 5-trifluoromethyl-N-[3(R or S)-[1-[5,6-dihydro-4-hydroxy-2-oxo-6(R or S)-(2-phenethyl)-6(R or S)-n-propyl-2H-pyran-3-yl]-propyl]-phenyl]-2-pyridine sulfonamide; 5-trifluoromethyl-N-[3(R)-[1-[5,6-dihydro-4-hydroxy-2-oxo-6(R)-(2-phenethyl)-6(R)-n-propyl-2H-pyran-3-yl]-propyl]-phenyl]-2-pyridine sulfonamide; (3R,6R)—N-[3-[1-(5,6-dihydro-4-hydroxy-2-oxo-6-(2-phenylethyl)-6-propyl-2H-pyran-3-yl)propyl]-phenyl]-5-(trifluoromethyl)-2-pyridinesulfonamide; 5-trifluoromethyl-N-[3(R)-[1-[5,6-dihydro-4-hydroxy-2-oxo-6(S)-(2-phenethyl)-6(S)-n-propyl-2H-pyran-3-yl]-propyl]-phenyl]-2-pyridinesulfonamide; (3R,6S)—N-[3-[1-(5,6-Dihydro-4-hydroxy-2-oxo-6-(2-phenylethyl)-6-propyl-2H-pyran-3-yl)propyl]-phenyl]-5-(trifluoromethyl)-2-pyridine sulfonamide; 5-trifluoromethyl-N-[3(S)-[1-[5,6-dihydro-4-hydroxy-2-oxo-6(R)-(2-phenethyl)-6(R)-n-propyl-2H-pyran-3-yl]-propyl]-phenyl]-2-pyridinesulfonamide; (3S,6R)—N-[3-[1-[5,6,-dihydro-4-hydroxy-2-oxo-6-(2-phenylethyl)-6-propyl-2H-pyran-3-yl]propyl]phenyl]-5-(trifluoromethyl)-2-pyridinesulfonamide; 5-trifluoromethyl-N-[3(S)-[1-[5,6-dihydro-4-hydroxy-2-oxo-6(S)-(2-phenethyl)-6(S)-n-propyl-2H-pyran-3-yl]-propyl]-phenyl]-2-pyridine sulfonamide; (3S,6S)—N-[3-[1-[5,6-dihydro-4-hydroxy-2-oxo-6-(2-phenylethyl)-6-propyl-2H-pyran-3-yl]propyl]phenyl]-5-(trifluoromethyl)-2-pyridinesulfonamide; N-[3-[1-(S)-[5,6,-dihydro-4-hydroxy-2-oxo-6-(2-phenylethyl)-6-propyl-2H-pyran-3-yl]propyl]phenyl]-5-(trifluoromethyl)-2-pyridinesulfonamide; (3R)—N-[3-[1-(5,6-dihydro-4-hydroxy-2-oxo-6-propyl-6-phenethyl-2H-pyran-3-yl)propyl]phenyl]-5-(trifluoromethyl)-2-pyridinesulfonamide; N-[3-{1(R or S)-(4-hydroxy-5,6-dihydro-2-oxo-6,6-dipropyl-2H-pyran-3-yl)-propyl]phenyl}-5-cyanopyridine-2-sulfonamide; N-[3-{1(R)-(4-Hydroxy-5,6-dihydro-2-oxo-6,6-dipropyl-2H-pyran-3-yl)-propyl}phenyl]-5-cyanopyridine-2-sulfonamide; N-[3-{1(S)-(4-hydroxy-5,6-dihydro-2-oxo-6,6-dipropyl-2H-pyran-3-yl)-propyl]phenyl}-5-cyaopyridine-2-sulfonamide; N-[3-{1(R or S)-(4-hydroxy-5,6-dihydro-2-oxo-6(R or S)-phenethyl-6-propyl-2H-pyran-3-yl)propyl]phenyl}-5-cyanopyridine-2-sulfonamide; N-[3-{1(R)-(4-Hydroxy-5,6-dihydro-2-oxo-6(R)-phenethyl-6-propyl-2H-pyran-3-yl)propyl}phenyl]-5-cyanopyridine-2-sulfonamide; N-[3-{1(R)-(4-hydroxy-5,6-dihydro-2-oxo-6(S)-phenethyl-6-propyl-2H-pyran-3-yl)propyl]phenyl}-5-cyanopyridine-2-sulfonamide; N-[3-{1(S)-(4-hydroxy-5,6-dihydro-2-oxo-6(R)-phenethyl-6-propyl-2H-pyran-3-yl)propyl]phenyl}-5-cyanopyridine-2-sulfonamide; N-[3-{1(S)-(4-hydroxy-5,6-dihydro-2-oxo-6(S)-phenethyl-6-propyl-2H-pyran-3-yl)propyl]phenyl}-5-cyanopyridine-2-sulfonamide; 5-amino-N-[3(R or S)-(1-[5,6-dihydro-4-hydroxy-2-oxo-6(R or S)-(2-phenylethyl)-6-propyl-2H-pyran-3-yl]propyl)phenyl]-2-pyridinesulfonamide; 5-amino-N-[3(R or S)-(1-[5,6-dihydro-4-hydroxy-2-oxo-6(R or S)-(2-phenylethyl)-6-propyl-2H-pyran-3-yl]propyl)phenyl]-2-pyridinesulfonamide; 5-trifluoromethyl-N-[3-[1-[4-hydroxy-2-oxo-6,6-di-n-propyl-5,6-dihydro-2H-pyran-3-yl]-propyl]-phenyl]-2-pyridinesulfonamide; N-[3-{1-(4-Hydroxy-5,6-dihydro-2-oxo-6,6-bis(2-phenylethyl)-2H-pyran-3-yl)propyl]phenyl}-5-cyanopyridine-2-sulfonamide; N-[3(R or S)-(1-[6,6-bis(2-phenylethyl)-5,6-dihydro-4-hydroxy-2-oxo-2H-pyran-3-yl]propyl)phenyl]-5-cyano-2-pyridinesulfonamide; N-[3(R or S)-(1-[6,6-bis(2-phenyl-ethyl)-5,6-dihydro-4-hydroxy-2-oxo-2H-pyran-3-yl]propyl)phenyl]-5-cyano-2-pyridinesulfonamide; N-[3-{1(R or S)-(4-hydroxy-5,6-dihydro-2-oxo-6,6-dipropyl-2H-pyran-3-yl)-propyl}phenyl]-5-aminopyridine-2-sulfonamide; N-[3-{1(S or R)-(4-hydroxy-5,6-dihydro-2-oxo-6,6-dipropyl-2H-pyran-3-yl)-propyl]phenyl}-5-aminopyridine-2-sulfonamide; 5-trifluoromethyl-N-[3-(R or S)-[1-[4-hydroxy-2-oxo-6,6-di-phenethyl-5,6-dihydro-2H-pyran-3-yl]-propyl]-phenyl]-2-pyridinesulfonamide; 5-trifluoromethyl-N-[3-(R or S)-[1-[4-hydroxy-2-oxo-6,6-di-phenethyl-5,6-dihydro-2H-pyran-3-yl]-propyl]-phenyl]-2-pyridinesulfonamide; N-[3-{1-(4-hydroxy-5,6-dihydro-2-oxo-6-phenethyl-6-propyl-2H-pyran-3-yl)propyl}phenyl]-5-cyanopyridine-2-sulfonamide; N-[3-{1-(4-Hydroxy-5,6-dihydro-2-oxo-6,6-dipropyl-2H-pyran-3-yl)propyl}phen yl]-5-cyanopyridine-2-sulfonamide; N-[3-{1-(4-hydroxy-5,6-dihydro-2-oxo-6,6-dipropyl-2H-pyran-3-yl)propyl}phenyl]-5-carbamoyl pyridine-2-sulfonamide; (3R,6R)—N-[3-[1-(5,6-dihydro-4-hydroxy-2-oxo-6-(2-phenylethyl)-6-propyl-2H-pyran-3-yl)propyl]phenyl]-5-(trifluoromethyl)-2-pyridinesulfonamide; (3R,6S)—N-[3-[1-(5,6-Dihydro-4-hydroxy-2-oxo-6-(2-phenylethyl)-6-propyl-2H-pyran-3-yl)propyl]phenyl]-5-(trifluoro methyl)-2-pyridinesulfonamide; (3S,6R)—N-[3-[1-[5,6,-dihydro-4-hydroxy-2-oxo-6-(2-phenylethyl)-6-propyl-2H-pyran-3-yl]propyl]phenyl]-5-(trifluoromethyl)-2-pyridine sulfonamide; (3S,6S)—N-[3-[1-[5,6-dihydro-4-hydroxy-2-oxo-6-(2-phenylethyl)-6-propyl-2H-pyran-3-yl]propyl]phenyl]-5-(trifluoromethyl)-2-pyridinesulfonamide; N-[3-[1-(S)-[5,6,-dihydro-4-hydroxy-2-oxo-6-(2-phenylethyl)-6-propyl-2H-pyran-3-yl]propyl]phenyl]-5-(trifluoromethyl)-2-pyridinesulfonamide; or (3R)—N-[3-[1-(5,6-dihydro-4-hydroxy-2-oxo-6-propyl-6-phenethyl-2H-pyran-3-yl)propyl]phenyl]-5-(trifluoromethyl)-2-pyridinesulfonamide.

In the preferred embodiment, the HIV protease inhibitor is nelfinavir, atazanavir, lopinavir, saquinavir or ritonavir, and is most preferably, nelfinavir or lopinavir.

The compounds for use in the present invention also includes racemates, racemic mixtures, individual diastereomers, and enantiomers of any of the above compounds, as well as pharmaceutically acceptable salts thereof. In addition, it is within the confines of the present invention that the methods, pharmaceutical product, and uses can include the combination of two or more HIV protease inhibitors. For example, the pharmaceutical composition can include the combination of an HIV protease inhibitor and a second HIV protease inhibitor such as ritonavir to, among other things, increase the bioavailability of the first HIV protease inhibitor. Such combinations can include, for example, nelfinavir and lopinavir, atazanavir and ritonavir, lopinavir and ritonavir, or saquinavir and ritonavir.

The HIV protease inhibitor compound for use in the present invention can be formulated in a pharmaceutical composition or a medicament with a pharmaceutically acceptable carrier for the treatment of systemic lupus erythematosus (SLE). In this regard, preferably the pharmaceutical composition is in unit dosage form, and most preferably is in unit dosage form for oral administration. In this regard, pharmaceutical compositions useful for these embodiments can be formulated without undue experimentation for administration to a subject as appropriate for the desired mode of administration. Additionally, proper dosages of the compositions can be determined without undue experimentation using standard dose-response protocols.

In an embodiment, the HIV protease inhibitor is administered to the subject at a dose of 200 mg to 2500 mg. In a preferred embodiment, the HIV protease inhibitor is adminstered to the subject at a dose of 500 mg to 2250 mg. In a most preferred embodiment, the HIV protease inhibitor is adminstered to the subject at 750 mg to 2250 mg, or 750 mg, or at approximately 750 mg, or 1250 mg, or at approximately 1250 mg, or 1500 mg, or at approximately 1500 mg, or 2250 mg, or at approximately 2250 mg. As used herein, the term “approximately” with regard to a referenced amount, means within ±10% of the stated referenced amount.

In an embodiment, the dose of HIV protease inhibitor is adminstered as needed. In an embodiment, the dose of HIV protease inhibitor is adminstered weekly, twice-weekly or thrice-weekly. In a preferred embodiment, the dose of HIV protease inhibitor is adminstered daily or a plurality of times each day. In a most preferred embodiment, the dose of HIV protease inhibitor is adminstered daily, twice daily, three times daily, or four times daily.

In non-limiting examples, the dose of HIV protease inhibitor may be administered in single unit dosage form or may be comprised of multiple unit dosage forms. In a preferred embodiment, the HIV protease inhibitor is nelfinavir and is adminstered in oral form as tablets comprising 250 mg or 625 mg active ingredient or as a dissolvable powder of 50 mg/g strength. In an embodiment, the nelfinavir is adminstered in a total daily dose of 250 mg, 500 mg, 750 mg, 1000 mg, 1250 mg, 1500 mg, 1750 mg, 2000 mg, 2250 mg or 2500 mg. In a preferred embodiment, the neflanivir is adminstered in a total daily dose of 750 mg or 1250 mg or 1500 mg or 2250 mg. In an embodiment, where the HIV protease inhibitor is nelfinavir, the nelfinavir in the tablet is in free base form. In a preferred embodiment, the nelfinavir in the tablet is a mesylate salt.

Any acceptable route of administration can be used. Pharmaceutical compositions designed, for example, for oral, lingual, sublingual, buccal and intrabuccal administration can be made without undue experimentation by means well known in the art, for example with an inert diluent or with an edible carrier. The compositions may be enclosed in gelatin capsules or compressed into tablets. In a preferred embodiment, the HIV protease inhibitor is administered orally. For the purpose of oral therapeutic administration, the pharmaceutical compositions of the present invention may be incorporated with excipients. Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

In a preferred embodiment, the HIV protease inhibitor is administered orally. In an embodiment where the HIV protease inhibitor is nelfinavir and it is being administered in pill form. Nelfinavir is commercially available from Pfizer (Agouron Pharmaceuticals) in pill form at 250 mg of active ingredient and at 625 mg of active ingredient. In an embodiment, the nelfinavir is in oral powder form for oral administration. Nelfinavir is commercially available from Pfizer (Agouron Pharmaceuticals) in a 50 mg/g strength (as nelfinavir free base).

Pharmaceutical compositions useful for the present invention can also be administered parenterally such as, for example, by intravenous, intramuscular, intrathecal or subcutaneous injection. Parenteral administration can be accomplished by incorporating the compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfate and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical compositions into the rectum or large intestine. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

Nasal administration includes administering the composition to the mucous membranes of the nasal passage or nasal cavity of the patient. Pharmaceutical compositions for nasal administration include compositions prepared by well-known methods to be administered, for example, as a nasal spray, nasal drop, suspension, gel, ointment, cream or powder. Administration of the composition may also take place using a nasal tampon or nasal sponge.

In an embodiment of the methods, the HIV protease inhibitor can be administered prophylactically. In an embodiment of the methods, the HIV protease inhibitor can be administered to prevent an SLE flare, or to reduce the extent or duration of an SLE flare, or to reduce the frequency of SLE flares. In an embodiment of the methods, the administering comprises subject self-administration of the HIV protease inhibitor.

In a preferred embodiment of the methods, the subject is diagnosed as having SLE prior to administration of the HIV protease inhibitor. SLE can be diagnosed by any method known in the art. In an embodiment, diagnosis of the subject as having SLE comprises detection of anti-double stranded DNA antibodies in a sample obtained from the subject. In an embodiment, diagnosis of the subject as having SLE comprises detection of anti-double stranded DNA antibodies in a serum sample obtained from the subject or in a cerebrospinal fluid sample obtained from the subject. In a most preferred embodiment, the anti-double stranded DNA antibodies are of the IgG class. In a most preferred embodiment, the sample from the subject is positive for anti-dsDNA antibodies at ≧30 units/ml. According to the methods of the invention, the subject can also be diagnosed as having SLE by being determined as having a modified SELENA-SLEDAI score of less than 6 or equal to 6, or an equivalent score on another SLE disease activity index. The modified SELENA-SLEDAI, or Systemic Lupus Erythematosus Disease Activity Index SELENA Modification, is a clinically used assessment and is widely available in the field, for example, at the American College of Rheumatology website, www.rheumatology.org. Other accepted SLE disease activity indexes may be used in place of the modified SELENA-SLEDAI, for example, European Consensus Lupus Activity Measure (ECLAM), Systemic Lupus Erythematosus Disease Activity Index (SLEDAI), Systemic Lupus Erythematosus Activity Measure (SLAM), or British Isles Lupus Assessment Group (BILAG). According to the methods of the invention, the subject can also be diagnosed as having SLE by both (i) being determined as having a modified SELENA-SLEDAI score of less than 6 or equal to 6, or an equivalent score on another SLE disease activity index, and (ii) by detection of anti-double stranded DNA antibodies in a sample obtained from the subject.

The methods disclosed herein can be used with any mammal having SLE. The uses of the HIV protease inhibitors herein can be used in treatment of any mammal having SLE. Preferably, the mammal is a human. In an embodiment, the mammal is not HIV positive. Preferably, the mammal is diagnosed with having SLE by detection of anti-dsDNA antibodies.

The present invention also provides a pharmaceutical product comprising an HIV protease inhibitor formulated in a pharmaceutically acceptable carrier; and a package insert providing instructions for the administration of the HIV protease inhibitor for the treatment of systemic lupus erythematosus (SLE). The HIV protease inhibitor can be formulated as appropriate for the desired mode of administration, and preferably is formulated for oral administration in unit dosage form. The pharmaceutical composition can include an HIV protease inhibitor or a combination of HIV protease inhibitors to increase bioavailability (e.g., by using ritonavir). For oral administration, the pharmaceutical product is preferably packaged as a bottle containing a preselected number of tablets, capsules or caplets. The package insert, in addition to including instructions for the administration of the HIV protease inhibitor or inhibitors for the treatment of systemic lupus erythematosus (SLE), may also include, for example, important safety information such as side effects of drug(s), contraindications, or instructions to take the drugs (e.g., the tablets, capsules or caplets) with a meal or within two hours after a meal.

All combinations of the various elements described herein are within the scope of the invention unless otherwise indicated herein or otherwise clearly contradicted by context.

This invention will be better understood from the Examples which follow. However, one skilled in the art will readily appreciate that the specific methods and results discussed are merely illustrative of the invention as described more fully in the claims which follow thereafter.

### Example 1

The DWEYS peptide and the peptidomimetic, Compound 12W (also known as FISLE412; 2-((4-amino-1-(4-(3-((tert-butylamino)methyl)octahydroisoquinolin-2(1H)-yl)-3-hydroxy-1-phenylbutan-2-ylamino)butan-2-ylamino)methyl)-1,2,3,4-tetrahydroquinolin-3-ol), inhibit the well-described lupus anti-dsDNA monoclonal antibody, R4A, from binding to known antigens (e.g., the DWEYS peptide itself, dsDNA, and the complement component C1q). A number of protease inhibitors, currently marketed as antiviral drugs to treat infection of human immunodeficiency virus, share structural features with both the DWEYS peptide and Compound 12W. To test whether these antiviral drugs might inhibit R4A binding, competitive ELISAs, with bound DWEYS peptide, bound dsDNA, or bound C1q protein, were performed. As shown in Table 1, all antiviral drugs demonstrated some activity to inhibit lupus anti-dsDNA antibody binding, and several are more potent than Compound 12W. Activity in these ELISAs is predictive of binding activity with naturally occurring anti-dsDNA antibodies in lupus patients. Thus, the antiviral drugs will be useful inhibitors of antibody-mediated pathogenic mechanisms in lupus, including neurotoxic and nephrotoxic mechanisms.

ELISAs. The ELISAs were performed essentially as described (DeGiorgio et al., 2001; Kowal et al., 2006). Antigens were absorbed to microtiter plate wells (either D-DWEYS (20 μg/ml) in PBS, calf thymus DNA (100 μg/ml) in NaHCO₃ (0.1M, pH 8.6) or full length complement component C1q (10-20 μg/ml) in PBS) overnight at 37° C. (Costar microtiter plates, Catalog #3690, Costar, Corning, N.Y.). The competitive ELISA was performed as described, using R4A monoclonal antibodies (2-20 μg/ml). Antibody preparations were pre-incubated with dilutions of Compound 12W or the listed antiviral drugs at various concentrations for 1 h at 37° C. and then transferred to the 96-well plates for a 1 h incubation at 37° C. The plates were washed and alkaline phosphatase-conjugated goat anti-mouse IgG antibody was added for 1 h at 37° C. Assays were developed at room temperature using p-nitrophenyl phosphate disodium, essentially as described (Gaynor et al., 1997; DeGiorgio et al., 2001; Kowal et al., 2006). The percent inhibition was calculated based on the OD405 nm: ((OD of test antibody, R4A, alone minus OD of test antibody and compound)/OD of test antibody alone)*100 at the most effective concentration of compound.

TABLE 1

| | 12W | Atazanavir | Lopinavir | Saquinavir | Ritonavir | Indinavir | Darunavir |
|---|---|---|---|---|---|---|---|
| DWEYS | 31% | 46% | 56% | 33% | 8% | 31% | 16% |
| DNA | 32% | 38% | 32% | 31% | 0 | 0 | 3% |
| C1q assay | 18% | 64% | 38% | 54% | 13% | 0 | 0 |

(% - inhibition of R4A binding)

### Example 2

Like the positive control peptidomimetic (12W), nelfinavir was found to inhibit the mouse monoclonal anti-dsDNA antibody, R4A, from binding to DWEYS peptide on ELISAs. Significant inhibition of R4A (5 μg/ml) binding was observed in the presence of nelfinavir at 25 μM and 50 μM. In early tests, inhibition of lupus patient sera components binding to DNA by nelfinavir was observed (data not shown). Sera, collected from lupus patients with moderately active disease and elevated titers of anti-dsDNA antibodies, was used for an ELISA assay for binding to DNA. The addition of nelfinavir in micromolar concentrations of 1, 10 and 100 decreased DNA binding by an average of 43%.

In addition, improved inhibition of R4A from binding to D-DWEYS-5 peptide on ELISAs by 1 μM, 10 μM and 100 μM each of atazanvir, lopinavir and saquniavir was found in experiments performed with 2 μg/ml R4A, with the highest concentrations of the HIV protease inhibitors showing the greatest inhibition.

Also, like the positive control peptidomimetic (12W), nelfinavir inhibited the mouse monoclonal anti-dsDNA antibody, R4A, from binding to DNA on ELISAs. Significant inhibition of R4A (5 μg/ml) binding in the presence of 25 μM and 50 μM nelfinavir was observed.

### Example 3

Nephrotoxicity: nelfinavir was also found to suppress pathologic deposition of mouse monoclonal anti-dsDNA antibody, R4A, to kidney glomeruli ex vivo. Of the four HIV protease inhibitors tested (lopinavir, atazanavir and saquinavir being the others), only nelfinavir decreased the R4A binding detected with fluorescently-labeled secondary antibody. For methodology see Bloom, O. et al., (2011), PNAS 108:10255-10259 and Zhang, J. et al., (2009), J. Autoimmun. 33:270-274.

### Example 4

Neurotoxicity: protection from R4A-induced apoptosis in the central nervous system was assessed by TUNEL staining. Lopinavir and nelfinavir were found to block neurotoxicity of mouse monoclonal anti-dsDNA antibody, R4A, in vivo. R4A was pre-incubated with vehicle or HIV protease inhibitors for 15 min. at 37° C. The preparation was then stereotaxically injected into the hippocampus of test animals. After 48 hours, brain slices at the injection site were subjected to TUNEL staining. Injection of R4A with vehicle elicited neurotoxicity at regions around the CA1 hippocampal injection site as determined by TUNEL positive staining. In contrast, neurons were protected when autoantibodies were pre-incubated with either lopinavir or nelfinavir. For methodology see Bloom, O. et al., (2011), supra, and Zhang, J. et al., (2009), supra.

### Example 5

A clinical trial is performed to confirm the immunomodulatory properties of an HIV protease inhibitor (PI), preferably of nelfinavir, in Systemic Lupus Erythematosus (SLE). The safety and tolerability of nelfinavir is determined in patients with SLE that have stable disease activity and elevated titers of anti-dsDNA antibodies. Additionally, the inhibitory effects of nelfinavir on anti-dsDNA antibodies is explored.

A Phase I, double-blind, placebo-controlled dose escalation study is carried out to demonstrate that the protease inhibitor nelfinavir will significantly decrease anti-dsDNA autoantibody binding. Secondary objectives include to assess disease activity; to assess the safety and tolerability of nelfinavir in SLE patients; to assess the effect of nelfinavir on serum cytokine levels (IL-1, IL-6, and TNF); to assess the effect of nelfinavir on serum levels of C3, C4 and CRP; to assess the effect of nelfinavir on the interferon signature; and to assess the effect of nelfinavir on serum anticardiolipin-binding activity.

Tablets of nelfinavir are commercially available from Pfizer (Agouron Pharmaceuticals) for oral administration as a light blue, capsule-shaped tablet with a clear film coating in 250 mg strength (as nelfinavir free base) and as a white oval tablet with a clear film coating in 625 mg strength (as nelfinavir free base). Each tablet contains the following common inactive ingredients: calcium silicate, crospovidone, magnesium stearate, hypromellose, and triacetin. The 250 mg tablet contains FD&C blue #2 powder and the 625 mg tablet contains colloidal silicon dioxide. Nelfinavir oral powder is available for oral administration in a 50 mg/g strength (as nelfinavir free base) in bottles. The oral powder contains the following inactive ingredients: microcrystalline cellulose, maltodextrin, dibasic potassium phosphate, crospovidone, hypromellose, aspartame, sucrose palmitate, and natural and artificial flavor. Nelfinavir has a plasma half life of approximately 3.5 to 5 hours and a steady state of the drug is achieved in 6 days. It is metabolized by several cytochrome P-450 enzymes including CYP3A4, CYP2C19, CYP2C9, and CYP2D6.

The trial is a randomized, double-blind, placebo-controlled, Phase I, dose escalation study which is designed to assess the effect of nelfinavir on dsDNA binding in patients with stable SLE and a modified SELENA-SLEDAI score of less than or equal to 6 and positive for anti-ds DNA antibody at the time of screening. Three cohorts of subjects are planned. Within each cohort some subjects will receive active drug and the remaining subjects will receive placebo. The doses of nelfinavir for cohorts 1, 2, and 3 will be 750 mg orally once daily, twice daily and three times daily, respectively. The total duration of the study for each subject will be up to 10 weeks. Subjects will be screened and if eligible will return within a 28 day window for randomization and initiation of treatment (Day 0). Study medication will be administered for a 14 day treatment period which will then be followed by a 4 week safety follow-up period. A Data and Safety Monitoring Board (DSMB) will review blinded safety data from all study visits within each cohort and determine whether or not it is appropriate for enrollment to proceed into the next cohort.

Trial Schedule—Day 0 (initiation of treatment): Subjects meeting the eligibility criteria will be randomized to receive active drug or placebo once, twice or three times a day, depending on the cohort. Medical history and current medications will be reviewed and updated. Subjects will undergo a physical examination. Prior to receiving the first dose of study drug, blood will be obtained for anti-Smith/anti-RNP, anti-Ro/anti-La antibodies, anti-cardiolipin antibodies, CRP, cytokine levels, urinalysis and anti-DNA binding. Disease activity will be assessed using the modified SELENA-SLEDAI. Blood will additionally be obtained for the mechanistic studies which will include cytokine levels, anti-DNA binding inhibition, peptide binding inhibition and the interferon signature. Day 6: All subjects will return on Day 6 for assessment of adverse events and medication review and mechanistic and basic laboratory evaluation (CBC with differential, comprehensive chemistry, urinalysis, anti-DNA binding inhibition, peptide binding inhibition). Day 13 (last day of study drug): Subjects will return on Day 13 for assessment of adverse events, medication review and physical exam and assessment of disease activity. The following labs will be drawn at this visit: CBC with differential, comprehensive metabolic panel, urinalysis, C3, C4, anti-dsDNA, anti-cardiolipin, CRP and a fasting lipid profile. Blood will also be drawn for the mechanistic studies that accompany this protocol (cytokine levels, anti-DNA binding inhibition, peptide binding inhibition and the interferon signature). Day 28 (2 weeks after stopping study drug): Subjects will return for assessment of adverse events, medication review and disease activity (MODIFIED SELENA-SLEDAI). Labs will be sent for CBC, comprehensive metabolic panel, C3, C4, anti-dsDNA antibody, anti-cardiolipin antibody, and urinalysis. Blood will also be drawn for the mechanistic studies that accompany this protocol (cytokine levels, anti-DNA binding inhibition, peptide binding inhibition and the interferon signature). Day 42 (end of study): Subjects will return for review of adverse events and safety follow-up. Including a basic laboratory evaluation (CBC with differential, comprehensive chemistry, urinalysis).

Primary endpoint: The primary endpoint is a mechanistic endpoint—inhibition of anti-dsDNA binding. Inhibition of anti-dsDNA binding will be determined by assessing changes in anti-ds DNA antibody titers from baseline to Day 13.

Safety will be evaluated by determining the frequency of adverse events and serious adverse events including measurements of hematology, clinical chemistry, and urinalysis parameters over the course of the study from baseline to day 42. The primary safety endpoint will be the proportion of subjects in each study arm experiencing any adverse event >Grade 3 (National Cancer Institute—Common Terminology Criteria [NCICTCAE]) over the duration of follow-up.

Disease activity—the study is exploratory and is not designed to assess efficacy as subjects are required to have stable disease activity at enrollment. The effect of nelfinavir on clinical indicators of disease will be evaluated descriptively. Clinical indicators of disease will include: changes in serum C3 and C4 complement levels from baseline to Day 13; changes in serum CRP from baseline to Day 13; changes in the modified SELENA-SLEDAI (total score) from baseline to Day 13; and changes in serum anti-cardiolipin binding activity.

The clinical indicators of disease will be determined for each treatment group at each dose level. Interferon signature—secondary analyses will explore the potential effect of nelfinavir on the expression of the IFN signature. Changes in expression of IFN inducible genes will be determined from baseline to Day 13. Expression of IFN inducible genes will be determined for each treatment group at each dose level. Cytokines—changes in serum cytokine levels (IL-1, IL-6, and TNF) from baseline to Day 13 will be determined for each treatment group at each dose level.

Statistical Methods—this will be a randomized, double-blind, dose-escalation, placebo controlled, Phase I study designed to assess the immunomodulatory properties as well as safety and tolerability of nelfinavir in patients with SLE that have stable disease activity and elevated titers of anti-dsDNA antibodies. All patients will be grouped according to the treatment actually received. All patients who receive any amount of study drug will be included in the analyses; all placebo-treated patients at different dose levels will be combined into one control group. All results will be presented separately for the control group and each of the active doses. Descriptive statistics on continuous measurements will include means, medians, standard deviations, and ranges, while categorical data will be summarized using frequency counts and percentages. As such, the treatment groups will be summarized and analyzed by including 4 groups: nelfinavir 750 mg daily, 750 mg twice daily, 750 mg three times daily, and Placebo.

### LIST OF REFERENCES

DeGiorgio, L. A., et al. A subset of lupus anti-DNA antibodies cross-reacts with the NR2 glutamate receptor in systemic lupus erythematosus. Nat Med 7, 1189-1193 (2001).
Gaynor, B., et al. Peptide inhibition of glomerular deposition of an anti-DNA antibody. Proc Natl Acad Sci USA 94, 1955-1960 (1997).
Kowal, C., et al. Human lupus autoantibodies against NMDA receptors mediate cognitive impairment. Proc Natl Acad Sci USA 103, 19854-19859 (2006).

U.S. Pat. No. 5,849,911.

U.S. Pat. No. 5,914,332.

U.S. Pat. No. 5,196,438.

U.S. Pat. No. 5,541,206.

U.S. Pat. No. 5,648,497.

U.S. Pat. No. 5,413,999.

U.S. Pat. No. 5,843,946.

U.S. Pat. No. 5,484,926.

U.S. Pat. No. 5,585,397.

U.S. Pat. No. 6,436,989.

U.S. Pat. No. 5,582,195.

1. A method of treating systemic lupus erythematosus (SLE) in a subject comprising administering to the subject an amount of an HIV protease inhibitor effective to treat SLE.

2. The method of claim 1, wherein the HIV protease inhibitor is nelfinavir.

3. The method of claim 1, wherein the HIV protease inhibitor is atazanavir.

4. The method of claim 1, wherein the HIV protease inhibitor is lopinavir.

5. The method of claim 1, wherein the HIV protease inhibitor is sequinavir.

6. The method of claim 1, wherein the HIV protease inhibitor is ritonavir.

7. The method of claim 1, wherein the HIV protease inhibitor is indinavir.

8. The method of claim 1, wherein the HIV protease inhibitor is darunavir.

9. The method of claim 1, wherein a combination of HIV protease inhibitors is administered.

10. The method of claim 9, wherein the combination of HIV protease inhibitors comprises ritonavir and another HIV protease inhibitor or nelfinavir and another HIV protease inhibitor.

11. The method of claim 1, wherein the subject is diagnosed as having SLE prior to administration of the HIV protease inhibitor.

12.-16. (canceled)

17. A pharmaceutical product comprising (i) an HIV protease inhibitor formulated in a pharmaceutically acceptable carrier and (ii) a package insert providing instructions for the administration of the HIV protease inhibitor for the treatment of systemic lupus erythematosus (SLE).

18. The product of claim 17, wherein the HIV protease inhibitor is nelfinavir.

19. The product of claim 17, wherein the HIV protease inhibitor is atazanavir.

20. The product of claim 17, wherein the HIV protease inhibitor is lopinavir.

21. The product of claim 17, wherein the HIV protease inhibitor is saquinavir.

22. The product of claim 17, wherein the HIV protease inhibitor is ritonavir.

23. The product of claim 17, wherein the HIV protease inhibitor is indinavir.

24. (canceled)

25. The product of claim 17, which comprises a combination of HIV protease inhibitors.

26. The product of claim 17, wherein the combination of HIV protease inhibitors comprises ritonavir and another HIV protease inhibitor or nelfinavir and another HIV protease inhibitor.

27.-48. (canceled)
